(19) United States
(12) Patent Application Publication
(10) Pub. No.: US 2008/0281119 A1
(43) Pub. Date: Nov. 13, 2008

(54) Separation of Nickel(0) Complexes and Phosphorus-Containing Ligands from Nitrile Mixtures

(75) Inventors: Jens Scheidel, Hirschberg (DE); Tim Jungkamp, Kapellen (BE); Michael Bartsch, Neustadt (DE); Gerd Haderlein, Grunstadt (DE); Robert Baumann, Mannheim (DE); Hermann Luyken, Ludwigshafen (DE); Petra Deckert, Wiesloch (DE); Peter Pfab, Bad Durkheim (DE); Wolfgang Siegel, Limburgerhof (DE); Tobias Aechtner, Mannheim (DE)

Correspondence Address: CONNOLLY BOVE LODGE & HUTZ LLP, 1875 EYE STREET, N.W., SUITE 1100, WASHINGTON, DC, 20006, US

(73) Assignee: BASF Aktiengesellschaft, Ludwigshafen (DE)

(21) Appl. No.: 10/586,493

(22) PCT Filed: Jan. 27, 2005
(86) PCT No.: PCT/EP2005/000779

§ 371 Date: Jul. 20, 2006

(30) Foreign Application Priority Data
Jan. 29, 2004 (DE) 102004004685.9
Sep. 15, 2004 (DE) 102004045036.6

(51) Int. Cl. C07C 253/34

(52) U.S. Cl. 558/308

(57) ABSTRACT

A process for extractively removing nickel(0) complexes having phosphorus ligands and/or free phosphorus ligands from a reaction effluent of a hydrocyanation of unsaturated mononitriles to dinitriles by extraction by means of a hydrocarbon, a phase separation of the hydrocarbon and of the reaction effluent into two phases being effected at a temperature T (in ° C.),

wherein the content of nickel(0) complexes having phosphorus ligands and/or free phosphorus ligands in the reaction effluent of the hydrocyanation, depending on the temperature T, is at least y % by weight and, irrespective of the temperature T, is a maximum of 60% by weight, where the numerical value of the minimum content y is given by the equation

y=0.5·T+20

and T is to be used in the equation as a dimensionless numerical value.

The invention relates to a process for extractively removing nickel(0) complexes having phosphorus ligands and/or free phosphorus ligands from a reaction effluent of a hydrocyanation of unsaturated mononitriles to dinitriles by extraction by means of a hydrocarbon, a phase separation of the hydrocarbon and of the reaction effluent into two phases being effected at a temperature T (in ° C.),

wherein the content of nickel(0) complexes having phosphorus ligands and/or free phosphorus ligands in the reaction effluent of the hydrocyanation, depending on the temperature T, is at least y % by weight and, irrespective of the temperature T, is a maximum of 60% by weight, where the numerical value of the minimum content y is given by the equation

y=0.5·T+20

and T is to be used in the equation as a dimensionless numerical value.

For hydrocyanations of unsaturated mononitriles, nickel complexes of phosphorus ligands are suitable catalysts. For example, adiponitrile, an important intermediate in nylon production, is prepared by double hydrocyanation of 1,3-butadiene. In a first hydrocyanation, 1,3-butadiene is reacted with hydrogen cyanide in the presence of nickel(0) which is stabilized with phosphorus ligands to give 3-pentenenitrile. In a second hydrocyanation, 3-pentenenitrile is subsequently reacted with hydrogen cyanide to give adiponitrile, likewise over a nickel catalyst, but, if appropriate, with addition of a Lewis acid and possibly of a promoter. Nickel(0) or Ni(0) mean nickel in the 0 oxidation state.

In order to increase the economic viability of the hydrocyanation, the nickel catalyst is typically removed and recycled (catalyst circulation). Since the catalyst system in the second hydrocyanation, which is a mixture of complex and free ligand, cannot be thermally stressed to a high degree, the high-boiling adiponitrile cannot be removed from the catalyst system by distillation. Therefore, the separation is generally carried out extractively using cyclohexane or methylcyclohexane as the extractant. The catalyst system remains, ideally fully, under real conditions at least partly, in the lighter cyclohexane or methylcyclohexane phase, while the heavier phase is more polar and comprises crude adiponitrile and, where present, the Lewis acid. After the phase separation, the extractant is removed generally by distillation under reduced pressure. The boiling pressure of the extractant is distinctly higher than that of the adiponitrile.

U.S. Pat. Nos. 3,773,809 and 5,932,772 describe the extraction of the catalyst complex and of the ligands with paraffins and cycloparaffins, for example cyclohexane, heptane and octane, or alkylaromatics.

U.S. Pat. No. 4,339,395 discloses a process for extractively working up reaction effluents of hydrocyanations for catalyst systems having monodentate ligands and a triarylborane as a promoter, in which a small amount of ammonia is metered in in order to prevent rag formation.

WO 2004/062765 describes the extractive removal of a nickel diphosphite catalyst from a mixture of mono- and dinitriles with alkanes or cycloalkanes as an extractant, wherein the mixture is treated with a Lewis base, for example organoamines or ammonia.

U.S. Pat. No. 5,847,191 discloses a process for the extractive workup of reaction effluents of hydrocyanations, wherein the chelate ligands bear C₉- to C₄₀-alkyl radicals.

U.S. Pat. No. 4,990,645 states that the extractability of the nickel complex and of the free ligands can be improved when the Ni(CN)₂ solid formed in the reaction is removed in a decanter before the extraction. To this end, a portion of the pentene nitrile is evaporated off beforehand in order to reduce the solubility of the catalyst and of the Ni(CN)₂.

In order to achieve a phase separation between cyclohexane or methylcyclohexane phase and the crude adiponitrile-containing phase, it has hitherto been necessary to achieve a minimum conversion of 3-pentenenitrile. For instance, U.S. Pat. No. 3,773,809 requires a minimum conversion of the 3-pentenenitrile of 60% as a condition for the phase separation when cyclohexane is used as the extractant, so that the ratio between 3-pentenenitrile and adiponitrile is below 0.65. When this ratio is not achieved by conversion of 3-pentenenitrile, either 3-pentenenitrile has to be preevaporated or adiponitrile has to be added in order to come to a ratio of below 0.65. A problem with this minimum conversion of 3-pentenenitrile is that a higher degree of conversion of 3-pentenenitrile is associated with a poorer selectivity for adiponitrile based on 3-pentenenitrile and hydrogen cyanide. Furthermore, a minimum conversion of the 3-pentenenitrile of 60% leads to a lower lifetime of the catalyst system.

It is therefore an object of the present invention to remedy the aforementioned disadvantages, i.e. to provide a process for extractively removing nickel(0) complexes having phosphorus ligands and/or free phosphorus ligands from a reaction effluent of a hydrocyanation of unsaturated mononitriles to dinitriles, which avoids the above-described disadvantages of the known processes. In particular, it should be possible in the process according to the invention to carry out the extractive removal of nickel(0) complexes having phosphorus ligands and/or free phosphorus ligands from a reaction effluent of a hydrocyanation, in which a lower conversion of unsaturated mononitriles has to be attained and in which a preevaporation of the unsaturated mononitrile or an addition of the dinitrile is not necessarily required.

Accordingly, the process specified at the outset has been found. Preferred embodiments of the invention can be taken from the subclaims.

In a particularly preferred embodiment, the process according to the invention is used in the preparation of adiponitrile. The process according to the invention is thus preferentially intended for 3-pentenenitrile as the mononitrile and adiponitrile as the dinitrile. Preference is likewise given to obtaining the reaction effluent of the hydrocyanation by reacting 3-pentenenitrile with hydrogen cyanide in the presence of at least one nickel(0) complex with phosphorus ligands, if appropriate in the presence of at least one Lewis acid (for example as the promoter).

### Process Principle

The process according to the invention is suitable for extractively removing Ni(0) complexes which contain phosphorus ligands and/or free phosphorus ligands from a reaction effluent which is obtained in a hydrocyanation of unsaturated mononitriles to dinitriles. These complexes are described below.

The reaction effluent is extracted by means of a hydrocarbon; in the course of this, a phase separation of the hydrocarbon and of the reaction effluent into two phases occurs at a temperature T (in ° C.). In general, a first phase which is enriched in the Ni(0) complexes or ligands mentioned compared to the reaction effluent, and a second phase which is enriched in dinitriles compared to the reaction effluent are formed. Usually, the first phase is the lighter phase, i.e. the upper phase, and the second phase the heavier phase, i.e. the lower phase.

According to the invention, the maximum content of nickel(0) complexes having phosphorus and/or free ligands in the reaction effluent of the hydrocyanation is 60% by weight. This maximum content is independent of the temperature T. The minimum content of the Ni(0) complexes or ligands mentioned is dependent upon T and is y % by weight, where the numerical value of the minimum content y is given by the equation

y=0.5·T+20

and T is used as a dimensionless numerical value. For example, when the temperature T of the phase separation is 50° C., y=0.5·50+20=45; the minimum content at T=50° C. is accordingly 45% by weight.

Depending on the phase ratio, the extraction has an extraction coefficient, defined as the ratio of the mass content of the nickel(0) complexes or ligands mentioned in the upper phase to the mass content of the nickel(0) complexes or ligands mentioned in the lower phase, for each theoretical extraction stage of preferably from 0.1 to 10, more preferably from 0.8 to 5. The extractive action, measured by the extraction coefficient for the free ligand, is equally good or better, preferably better than for the nickel(0) complex.

After the phase separation, the upper phase contains preferably between 50 and 99% by weight, more preferably between 60 and 97% by weight, in particular between 80 and 95% by weight, of the hydrocarbon used for the extraction.

The Lewis acid which is, if appropriate (specifically in the second hydrocyanation mentioned at the outset), present in the feed stream of the extraction remains preferably for the most part and more preferably fully in the lower phase. Here, fully means that the residual concentration of the Lewis acid in the upper phase is preferably less than 1% by weight, more preferably less than 0.5% by weight, in particular less than 500 ppm by weight.

### Hydrocarbon

The hydrocarbon is the extractant. It has a boiling point of preferably at least 30° C., more preferably at least 60° C., in particular at least 90° C., and preferably at most 140° C., more preferably at most 135° C., in particular at most 130° C., based in each case on a pressure of 10⁵ Pa absolute.

Particular preference is given to using a hydrocarbon, this referring in the context of the present invention either to an individual hydrocarbon or to a mixture of such hydrocarbons, for the removal, especially by extraction, of adiponitrile from a mixture comprising adiponitrile and the Ni(0)-containing catalyst, said hydrocarbon having a boiling point in the range between 90° C. and 140° C. The catalyst, if appropriate with addition of a suitable solvent which is higher-boiling than the hydrocarbon H (e.g. pentenenitrile), may advantageously be obtained by distillative removal of the hydrocarbon from the mixture obtained after the removal by this process, in which case the use of a hydrocarbon having a boiling point in the range specified permits a particularly economically viable and technically simple removal as a result of the possibility of condensing the hydrocarbon distilled off with river water.

Suitable hydrocarbons are described, for example, in U.S. Pat. No. 3,773,809, column 3, lines 50-62. Preference is given to a hydrocarbon selected from cyclohexane, methylcyclohexane, cycloheptane, n-hexane, n-heptane, isomeric heptanes, n-octane, isooctane, isomeric octanes such as 2,2,4-trimethylpentane, cis- and trans-decalin or mixtures thereof, especially of cyclohexane, methylcyclohexane, n-heptane, isomeric heptanes, n-octane, isomeric octanes such as 2,2,4-trimethylpentane, or mixtures thereof. Particular preference is given to using cyclohexane, methylcyclohexane, n-heptane or n-octane.

Very particular preference is given to n-heptane or n-octane. With these hydrocarbons, the undesired rag formation is particularly low. Rag refers to a region of incomplete phase separation between upper and lower phase, usually a liquid/liquid mixture in which solids may also be dispersed. Excess rag formation is undesired since it hinders the extraction and the extraction apparatus can under some circumstances be flooded by rag, as a result of which it can no longer fulfill its separation task.

The hydrocarbon used is preferably anhydrous, anhydrous meaning a water content of below 100 ppm by weight, preferably below 50 ppm by weight, in particular below 10 ppm by weight. The hydrocarbon may be dried by suitable processes known to those skilled in the art, for example by adsorption or azeotropic distillation. The drying may be effected by a step preceding the process according to the invention.

### Configuration of the Extraction.

The extraction of the nickel(0) complexes or ligands from the reaction effluent may be carried out in any suitable apparatus known to those skilled in the art, preferably in countercurrent extraction columns, mixer-settler units or combinations of mixer-settler units with columns. Particular preference is given to the use of countercurrent extraction columns which are equipped in particular with sheet metal packings as dispersing elements. In a further particularly preferred embodiment, the extraction is performed in countercurrent in a compartmented, stirred extraction column.

Regarding the dispersion direction, in a preferred embodiment of the process, the hydrocarbon is used as the continuous phase and the reaction effluent of the hydrocyanation as the disperse phase. This generally also shortens the phase separation time and reduces rag formation. However, the reverse dispersion direction is also possible, i.e. reaction effluent as the continuous and hydrocarbon as the disperse phase. The latter is especially true when the rag formation is reduced or suppressed fully by preceding solids removal (see below), higher temperatures in the extraction or phase separation or use of a suitable hydrocarbon. Typically, the dispersion direction more favorable for the separating performance of the extraction apparatus is selected.

In the extraction, a phase ratio of preferably from 0.1 to 10, more preferably from 0.4 to 2.5, in particular from 0.75 to 1.5, calculated in each case as the ratio of mass of the hydrocarbon added to mass of the mixture to be extracted, is used.

The absolute pressure during the extraction is preferably from 10 kPa to 1 MPa, more preferably from 50 kPa to 0.5 MPa, in particular from 75 kPa to 0.25 MPa (absolute).

The extraction is preferably carried out at a temperature of −15 to 120° C., in particular from 20 to 100° C. and more preferably from 30 to 80° C. It has been found that the rag formation is lower at a higher temperature of the extraction.

In a particularly preferred embodiment, the extraction is operated with a temperature profile. In particular, operation is effected in this case at an extraction temperature of at least 60° C., preferably from 60 to 95° C. and more preferably at least 70° C.

The temperature profile is preferably configured in such a way that, in that region of the extraction in which the content of nickel(0) complexes having phosphorus ligands and/or free phosphorus ligands is higher than in the other region, the temperature is lower than the other region. In this way, the thermally labile Ni(0) complexes are less thermally stressed and their decomposition is reduced.

Where an extraction column, for example, is used for the extraction and a temperature profile is employed, the lowest temperature is established at the top of the column and the highest at the bottom of the column. The temperature differential between top and bottom of the column may be, for example, from 0 to 30° C., preferably from 10 to 30° C. and in particular from 20 to 30° C.

### Configuration of the Phase Separation

Depending on the apparatus configuration, the phase separation may also be viewed in spatial terms and in terms of time as the last part of the extraction. For the phase separation, a wide pressure, concentration and temperature range may typically be selected, and the optimal parameters for the particular composition of the reaction mixture can be determined readily by a few simple preliminary experiments.

The temperature T in the phase separation is typically at least 0° C., preferably at least 10° C., more preferably at least 20° C. Typically, it is at most 120° C., preferably at most 100° C., more preferably at most 95° C. For example, the phase separation is carried out at from 0 to 100° C., preferably from 60 to 95° C. It has been found that the rag formation is lower at a higher temperature of the phase separation.

The pressure in the phase separation is generally at least 1 kPa, preferably at least 10 kPa, more preferably 20 kPa. In general, it is at most 2 MPa, preferably at most 1 MPa, more preferably at most 0.5 MPa absolute.

The phase separation time, i.e. the duration from the mixing of the reaction effluent with the hydrocarbon (extractant) to the formation of a uniform upper phase and a uniform lower phase may vary within wide limits. The phase separation time is generally from 0.1 to 60 min, preferably from 1 to 30 min and in particular from 2 to 10 min. When the process according to the invention is carried out on the industrial scale, a maximum phase separation time of 15 min, in particular 10 min, is typically technically and economically sensible.

It has been found that the phase separation time is reduced in an advantageous manner especially when long-chain aliphatic alkanes such as n-heptane or n-octane are used.

The phase separation may be carried out in one or more apparatuses, known to those skilled in the art, for such phase separations. In an advantageous embodiment, the phase separation may be carried out in the extraction apparatus, for example in one or more mixer-settler combinations or by equipping an extraction column with a calming zone.

In the phase separation, two liquid phases are obtained, of which one phase has a higher proportion of the Ni(0) complex having phosphorus ligands and/or free phosphorus ligands, based on the total weight of this phase, than the other phase or other phases.

In a preferred embodiment of the process, an adiponitrile content of the effluent stream from the hydrocyanation of greater than 30% by weight is established at a temperature of the phase separation of 20° C., and the content of nickel(0) complexes or ligands is less than 60% by weight, preferably less than 50% by weight, more preferably less than 40% by weight.

In a further preferred embodiment of the process, an adiponitrile content of the effluent stream from the hydrocyanation of greater than 40% by weight is established at a temperature of the phase separation of 40° C., and the content of nickel(0) complexes or ligands is less than 60% by weight, preferably less than 50% by weight, more preferably less than 40% by weight.

In a preferred embodiment of the process according to the invention, an adiponitrile content of the effluent stream from the hydrocyanation of greater than 50% by weight is established at a temperature of the phase separation of 60° C., and the content of nickel(0) complexes or ligands is less than 50% by weight, more preferably less than 40% by weight.

Optional Treatment with Ammonia or Amine

In a preferred embodiment of the process according to the invention, the reaction effluent of the hydrocyanation is treated before or during the extraction with ammonia or a primary, secondary or tertiary, aromatic or aliphatic amine. Aromatic includes alkylaromatic, and aliphatic includes cycloaliphatic.

It has been found that this ammonia or amine treatment can reduce the content of nickel(0) complex or ligand in the second phase enriched with dinitriles (usually lower phase), i.e. the distribution of Ni(0) complex or ligand between the two phases is shifted in favor of the first phase (upper phase). The ammonia or amine treatment improves the catalyst enrichment in the upper phase; this means lower catalyst losses in the catalyst cycle and increases the economic viability of the hydrocyanation.

Accordingly, in this embodiment, the extraction is preceded by a treatment of the reaction effluent with ammonia or an amine or this is effected during the extraction. The treatment during the extraction is less preferred.

The amines used are monoamines, diamines, triamines or more highly functional amines (polyamines). The monoamines typically have alkyl radicals, aryl radicals or arylalkyl radicals having from 1 to 30 carbon atoms; suitable monoamines are, for example, primary amines, e.g. monoalkylamines, secondary or tertiary amines, e.g. dialkylamines. Suitable primary monoamines are, for example, butylamine, cyclohexylamine, 2-methylcyclohexylamine, 3-methylcyclohexylamine, 4-methylcyclohexylamine, benzylamine, tetrahydrofurfurylamine and furfurylamine. Useful secondary monoamines are, for example, diethylamine, dibutylamine, di-n-propylamine and N-methylbenzylamine. Suitable tertiary amines are, for example, trialkylamines having C₁₋₁₀ alkyl radicals such as trimethylamine, triethylamine or tributylamine.

Suitable diamines are, for example, those of the formula R¹—NH—R²—NH—R³, where R¹, R² and R³ are each independently hydrogen or an alkyl radical, aryl radical or arylalkyl radical having from 1 to 20 carbon atoms. The alkyl radical may be linear or, especially for R², also cyclic. Suitable diamines are, for example, ethylenediamine, propylenediamines (1,2-diaminopropane and 1,3-diaminopropane), N-methyl-ethylenediamine, piperazine, tetramethylenediamine (1,4-diaminobutane), N,N′-dimethylethylenediamine, N-ethylethylenediamine, 1,5-diaminopentane, 1,3-diamino-2,2-diethylpropane, 1,3-bis(methylamino)propane, hexamethylenediamine(1,6-diaminohexane), 1,5-diamino-2-methylpentane, 3-(propylamino)propylamine, N,N′-bis(3-aminopropyl)piperazine, N,N′-bis(3-aminopropyl)piperazine and isophoronediamine (IPDA).

Suitable triamines, tetramines or more highly functional amines are, for example, tris(2-aminoethyl)amine, tris(2-aminopropyl)amine, diethylenetriamine (DETA), triethylenetetramine (TETA), tetraethylenepentamine (TEPA), isopropylenetriamine, dipropylenetriamine and N,N′-bis(3-aminopropylethylenediamine). Aminobenzylamines and aminohydrazides having 2 or more amino groups are likewise suitable.

Of course, it is also possible to use mixtures of ammonia with one or more amines, or mixtures of a plurality of amines.

Preference is given to using ammonia or aliphatic amines, in particular trialkylamines having from 1 to 10 carbon atoms in the alkyl radical, for example trimethylamine, triethylamine or tributylamine, and also diamines such as ethylenediamine, hexa-methylenediamine or 1,5-diamino-2-methylpentane.

Particular preference is given to ammonia alone; in other words, particular preference is given to using no amine apart from ammonia. Very particular preference is given to anhydrous ammonia; in this case, anhydrous means a water content below 1% by weight, preferably below 1000 ppm by weight and in particular below 100 ppm by weight.

The molar ratio of amine to ammonia may be varied within wide limits, and is generally from 10 000:1 to 1:10 000.

The amount of the ammonia or amine used depends, inter alia, on the type and amount of the nickel(0) catalyst and/or of the ligands and, if used, on the type and amount of the Lewis acid which is used as a promoter in the hydrocyanation. Typically, the molar ratio of ammonia or amine to Lewis acid is at least 1:1. The upper limit of this molar ratio is generally uncritical and is, for example, 100:1; however, the excess of ammonia or amine should not be so great that the Ni(0) complex or its ligand decomposes. The molar ratio of ammonia or amine to Lewis acid is preferably from 1:1 to 10:1, more preferably from 1.5:1 to 5:1, and in particular about 2.0:1. When a mixture of ammonia and amine is used, these molar ratios apply to the sum of ammonia and amine.

The temperature in the treatment with ammonia or amine is typically not critical and is, for example, from 10 to 140° C., preferably from 20 to 100° C. and in particular from 20 to 90° C. The pressure is generally not critical either.

The ammonia or the amine may be added to the reaction effluent in gaseous form, in liquid form (under pressure) or dissolved in a solvent. Suitable solvents are, for example, nitriles, especially those which are present in the hydrocyanation, and also aliphatic, cycloaliphatic or aromatic hydrocarbons, as used in the process according to the invention as extractants, for example cyclohexane, methylcyclohexane, n-heptane or n-octane.

The ammonia or amine addition is effected in customary apparatus, for example those for gas introduction or in liquid mixers. The solid which precipitates out in many cases may either remain in the reaction effluent, i.e. a suspension is fed to the extraction, or be removed as described below.

### Optional Removal of the Solids

In a preferred embodiment, the solids present in the reaction effluent are removed at least partly before the extraction. In many cases, this allows the extraction performance of the process according to the invention to be improved further. It is suspected that a high solids content hinders the mass transfer during the extraction, which makes necessary larger and thus more expensive extraction apparatus. It has also been found that the solids removal before the extraction often distinctly reduces or fully suppresses the undesired rag formation.

Preference is given to configuring the solids removal in such a way that the solid particles having a hydraulic diameter of greater than 5 μm, in particular greater than 1 μm and more preferably greater than 100 nm are removed.

For the solids removal, it is possible to use customary processes, for example filtration, crossflow filtration, centrifugation, sedimentation, classification or preferably decantation, for which common apparatus such as filters, centrifuges and decanters can be used.

Temperature and pressure in the solids removal are typically not critical. For example, it is possible to work within the aforementioned temperature and pressure ranges.

The solids removal may be effected before, during or after the optional treatment of the reaction effluent with ammonia or amine. The removal is preferably during or after the ammonia or amine treatment, more preferably thereafter.

When the solids are removed during or after the amine or ammonia treatment, the solids are usually compounds of ammonia or amine with the Lewis acid or the promoter used which are sparingly soluble in the reaction effluent. When, for example, ZnCl₂ is used, substantially sparingly soluble ZnCl₂.2NH₃ is formed in the ammonia treatment.

When the solids are removed before the ammonia or amine treatment, or if there is no treatment with ammonia or amine at all, the solids are generally nickel compounds of the +II oxidation state, for example nickel(II) cyanide or similar cyanide-containing nickel(II) compounds.

### Nickel(0) Complexes and Ligands

The Ni(0) complexes which contain phosphorus ligands and/or free phosphorus ligands are preferably homogeneously dissolved nickel(0) complexes.

The phosphorus ligands of the nickel(0) complexes and the free phosphorus ligands, which are removed by extraction in accordance with the invention, are preferably selected from mono- or bidentate phosphines, phosphites, phosphinites and phosphonites.

These phosphorus ligands preferably have the formula I

P(X¹R¹)(X²R²)(X³R³) (I).

In the context of the present invention, compound I is a single compound or a mixture of different compounds of the aforementioned formula.

According to the invention, X¹, X², X³ each independently are oxygen or a single bond. When all of the X¹, X² and X³ groups are single bonds, compound I is a phosphine of the formula P(R¹R²R³) with the definitions of R¹, R² and R³ specified in this description.

When two of the X¹, X² and X³ groups are single bonds and one is oxygen, compound I is a phosphinite of the formula P(OR¹)(R²)(R³) or P(R¹)(OR²)(R³) or P(R¹)(R²)(OR³) with the definitions of R¹, R² and R³ specified in this description.

When one of the X¹, X² and X³ groups is a single bond and two are oxygen, compound I is a phosphonite of the formula P(OR¹)(OR²)(R³) or P(R¹)(OR²)(OR³) or P(OR¹)(R²)(OR³) with the definitions of R¹, R² and R³ specified in this description.

In a preferred embodiment, all X¹, X² and X³ groups should be oxygen, so that compound I is advantageously a phosphite of the formula P(OR¹)(OR²)(OR³) with the definitions of R¹, R² and R³ specified in this description.

According to the invention, R¹, R², R³ are each independently identical or different organic radicals. R¹, R² and R³ are each independently alkyl radicals preferably having from 1 to 10 carbon atoms, such as methyl, ethyl, n-propyl, i-propyl, n-butyl, i-butyl, s-butyl, t-butyl, aryl groups such as phenyl, o-tolyl, m-tolyl, p-tolyl, 1-naphthyl, 2-naphthyl, or hydrocarbyl, preferably having from 1 to 20 carbon atoms, such as 1,1′-biphenol, 1,1′-binaphthol. The R¹, R² and R³ groups may be bonded together directly, i.e. not solely via the central phosphorus atom. Preference is given to the R¹, R² and R³ groups not being bonded together directly.

In a preferred embodiment, R¹, R² and R³ groups are radicals selected from the group consisting of phenyl, o-tolyl, m-tolyl and p-tolyl. In a particularly preferred embodiment, a maximum of two of the R¹, R² and R³ groups should be phenyl groups.

In another preferred embodiment, a maximum of two of the R¹, R² and R³ groups should be o-tolyl groups.

Particularly preferred compounds I which may be used are those of the formula Ia

(o-tolyl-O—)_{w}(m-tolyl-O—)ₓ(p-tolyl-O—)_{y}(phenyl-O—)_{z}P (Ia)

where w, x, y, z are each a natural number where w+x+y+z=3 and w, z≦2.

Such compounds I a are, for example, (p-tolyl-O—)(phenyl-O—)₂P, (m-tolyl-O—)(phenyl-O—)₂P, (o-tolyl-O—)(phenyl-O—)₂P, (p-tolyl-O—)₂(phenyl-O—)P, (m-tolyl-O—)₂(phenyl-O—)P, (o-tolyl-O—)₂(phenyl-O—)P, (m-tolyl-O—)(p-tolyl-O—)(phenyl-O—)P, (o-tolyl-O—)(p-tolyl-O—)(phenyl-O—)P, (o-tolyl-O—)(m-tolyl-O—)(phenyl-O—)P, (p-tolyl-O—)₃P, (m-tolyl-O—)(p-tolyl-O—)₂P, (o-tolyl-O—)(p-tolyl-O—)₂P, (m-tolyl-O—)₂(p-tolyl-O—)P, (o-tolyl-O—)₂(p-tolyl-O—)P, (o-tolyl-O—)(m-tolyl-O—)(p-tolyl-O—)P, (m-tolyl-O—)₃P, (o-tolyl-O—)(m-tolyl-O—)₂P, (o-tolyl-O—)₂(m-tolyl-O—)P or mixtures of such compounds.

For example, mixtures comprising (m-tolyl-O—)₃P, (m-tolyl-O—)₂(p-tolyl-O—)P, (m-tolyl-O—)(p-tolyl-O—)₂P and (p-tolyl-O—)₃P may be obtained by reacting a mixture comprising m-cresol and p-cresol, in particular in a molar ratio of 2:1, as obtained in the distillative workup of crude oil, with a phosphorus trihalide, such as phosphorus tri-chloride.

In another, likewise preferred embodiment, the phosphorus ligands are the phosphites, described in detail in DE-A 199 53 058, of the formula I b:

P(O—R¹)ₓ(O—R²)_{y}(O—R³)_{z}(O—R⁴)ₚ (I b)

where
R¹: aromatic radical having a C₁-C₁₈-alkyl substituent in the o-position to the oxygen atom which joins the phosphorus atom to the aromatic system, or having an aromatic substituent in the o-position to the oxygen atom which joins the phosphorus atom to the aromatic system, or having a fused aromatic system in the o-position to the oxygen atom which joins the phosphorus atom to the aromatic system,
R²: aromatic radical having a C₁-C₁₈-alkyl substituent in the m-position to the oxygen atom which joins the phosphorus atom to the aromatic system, or having an aromatic substituent in the m-position to the oxygen atom which joins the phosphorus atom to the aromatic system, or having a fused aromatic system in the m-position to the oxygen atom which joins the phosphorus atom to the aromatic system, the aromatic radical bearing a hydrogen atom in the o-position to the oxygen atom which joins the phosphorus atom to the aromatic system,
R³: aromatic radical having a C₁-C₁₈-alkyl substituent in the p-position to the oxygen atom which joins the phosphorus atom to the aromatic system, or having an aromatic substituent in the p-position to the oxygen atom which joins the phosphorus atom to the aromatic system, the aromatic radical bearing a hydrogen atom in the o-position to the oxygen atom which joins the phosphorus atom to the aromatic system,
R⁴: aromatic radical which bears substituents other than those defined for R¹, R² and R³ in the o-, m- and p-position to the oxygen atom which joins the phosphorus atom to the aromatic system, the aromatic radical bearing a hydrogen atom in the o-position to the oxygen atom which joins the phosphorus atom to the aromatic system,
x: 1 or 2,
y,z,p: each independently 0, 1 or 2, with the proviso that x+y+z+p=3.

Preferred phosphites of the formula I b can be taken from DE-A 199 53 058. The R¹ radical may advantageously be o-tolyl, o-ethylphenyl, o-n-propylphenyl, o-isopropyl-phenyl, o-n-butylphenyl, o-sec-butylphenyl, o-tert-butylphenyl, (o-phenyl)phenyl or 1-naphthyl groups.

Preferred R² radicals are m-tolyl, m-ethylphenyl, m-n-propylphenyl, m-isopropylphenyl, m-n-butylphenyl, m-sec-butylphenyl, m-tert-butylphenyl, (m-phenyl)phenyl or 2-naphthyl groups.

Advantageous R³ radicals are p-tolyl, p-ethylphenyl, p-n-propylphenyl, p-isopropyl-phenyl, p-n-butylphenyl, p-sec-butylphenyl, p-tert-butylphenyl or (p-phenyl)phenyl groups.

The R⁴ radical is preferably phenyl. p is preferably zero. For the indices x, y and z and p in compound I b, there are the following possibilities:

| x | y | z | p |
|---|---|---|---|
| 1 | 0 | 0 | 2 |
| 1 | 0 | 1 | 1 |
| 1 | 1 | 0 | 1 |
| 2 | 0 | 0 | 1 |
| 1 | 0 | 2 | 0 |
| 1 | 1 | 1 | 0 |
| 1 | 2 | 0 | 0 |
| 2 | 0 | 1 | 0 |
| 2 | 1 | 0 | 0 |

Preferred phosphites of the formula I b are those in which p is zero, and R¹, R² and R³ are each independently selected from o-isopropylphenyl, m-tolyl and p-tolyl, and R⁴ is phenyl.

Particularly preferred phosphites of the formula I b are those in which R¹ is the o-isopropylphenyl radical, R² is the m-tolyl radical and R³ is the p-tolyl radical with the indices specified in the table above; also those in which R¹ is the o-tolyl radical, R² is the m-tolyl radical and R³ is the p-tolyl radical with the indices specified in the table; additionally those in which R¹ is the 1-naphthyl radical, R² is the m-tolyl radical and R³ is the p-tolyl radical with the indices specified in the table; also those in which R¹ is the o-tolyl radical, R² is the 2-naphthyl radical and R³ is the p-tolyl radical with the indices specified in the table; and finally those in which R¹ is the o-isopropylphenyl radical, R² is the 2-naphthyl radical and R³ is the p-tolyl radical with the indices specified in the table; and also mixtures of these phosphites.

Phosphites of the formula I b may be obtained by
a) reacting a phosphorus trihalide with an alcohol selected from the group consisting of R¹OH, R²OH, R³OH and R⁴OH or mixtures thereof to obtain a dihalophosphorous monoester,
b) reacting the dihalophosphorous monoester mentioned with an alcohol selected from the group consisting of R¹OH, R²OH, R³OH and R⁴OH or mixtures thereof to obtain a monohalophosphorous diester and
c) reacting the monohalophosphorous diester mentioned with an alcohol selected from the group consisting of R¹⁰H, R²OH, R³OH and R⁴OH or mixtures thereof to obtain a phosphite of the formula I b.

The reaction may be carried out in three separate steps. Equally, two of the three steps may be combined, i.e. a) with b) or b) with c). Alternatively, all of steps a), b) and c) may be combined together.

Suitable parameters and amounts of the alcohols selected from the group consisting of R¹⁰H, R²OH, R³OH and R⁴OH or mixtures thereof may be determined readily by a few simple preliminary experiments.

Useful phosphorus trihalides are in principle all phosphorus trihalides, preferably those in which the halide used is Cl, Br, I, in particular Cl, and mixtures thereof. It is also possible to use mixtures of identically or differently halogen-substituted phosphines as the phosphorus trihalide. Particular preference is given to PCl₃. Further details on the reaction conditions in the preparation of the phosphites I b and for the workup can be taken from DE-A 199 53 058.

The phosphites I b may also be used in the form of a mixture of different phosphites I b as a ligand. Such a mixture may be obtained, for example, in the preparation of the phosphites I b.

However, preference is given to the phosphorus ligand being multidentate, in particular bidentate. The ligand used therefore preferably has the formula II

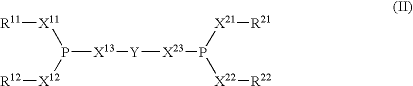

where
X¹¹, X¹², X¹³ X²¹, X²², X²³ are each independently oxygen or a single bond
R¹¹, R¹² are each independently identical or different, separate or bridged organic radicals
R²¹, R²² are each independently identical or different, separate or bridged organic radicals,
Y is a bridging group.

In the context of the present invention, compound II is a single compound or a mixture of different compounds of the aforementioned formula.

In a preferred embodiment, X¹¹, X¹², X¹³, X²¹, X²², X²³ may each be oxygen. In such a case, the bridging group Y is bonded to phosphite groups.

In another preferred embodiment, X¹¹ and X¹² may each be oxygen and X¹³ a single bond, or X¹¹ and X¹³ each oxygen and X¹² a single bond, so that the phosphorus atom surrounded by X¹¹, X¹² and X¹³ is the central atom of a phosphonite. In such a case, X²¹, X²² and X²³ may each be oxygen, or X²¹ and X²² may each be oxygen and X²³ a single bond, or X²¹ and X²³ may each be oxygen and X²² a single bond, or X²³ may be oxygen and X²¹ and X²² each a single bond, or X²¹ may be oxygen and X²² and X²³ each a single bond, or X²¹, X²² and X²³ may each be a single bond, so that the phosphorus atom surrounded by X²¹, X²² and X²³ may be the central atom of a phosphite, phosphonite, phosphinite or phosphine, preferably a phosphonite.

In another preferred embodiment, X¹³ may be oxygen and X¹¹ and X¹² each a single bond, or X¹¹ may be oxygen and X¹² and X¹³ each a single bond, so that the phosphorus atom surrounded by X¹¹, X¹² and X¹³ is the central atom of a phosphonite. In such a case, X²¹, X²² and X²³ may each be oxygen, or X²³ may be oxygen and X²¹ and X²² each a single bond, or X²¹ may be oxygen and X²² and X²³ each a single bond, or X²¹, X²² and X²³ may each be a single bond, so that the phosphorus atom surrounded by X²¹, X²² and X²³ may be the central atom of a phosphite, phosphinite or phosphine, preferably a phosphinite.

In another preferred embodiment, X¹¹, X¹² and X¹³ may each be a single bond, so that the phosphorus atom surrounded by X¹¹, X¹² and X¹³ is the central atom of a phosphine. In such a case, X²¹, X²² and X²³ may each be oxygen, or X²¹, X²² and X²³ may each be a single bond, so that the phosphorus atom surrounded by X²¹, X²² and X²³ may be the central atom of a phosphite or phosphine, preferably a phosphine. The bridging group Y is preferably an aryl group which is substituted, for example by C₁-C₄-alkyl, halogen, such as fluorine, chlorine, bromine, halogenated alkyl, such as trifluoromethyl, aryl, such as phenyl, or is unsubstituted, preferably a group having from 6 to 20 carbon atoms in the aromatic system, in particular pyrocatechol, bis(phenol) or bis(naphthol).

The R¹¹ and R¹² radicals may each independently be identical or different organic radicals. Advantageous R¹¹ and R¹² radicals are aryl radicals, preferably those having from 6 to 10 carbon atoms, which may be unsubstituted or mono- or polysubstituted, in particular by C₁-C₄-alkyl, halogen, such as fluorine, chlorine, bromine, halogenated alkyl, such as trifluoromethyl, aryl, such as phenyl, or unsubstituted aryl groups.

The R²¹ and R²² radicals may each independently be identical or different organic radicals. Advantageous R²¹ and R²² radicals are aryl radicals, preferably those having from 6 to 10 carbon atoms, which may be unsubstituted or mono- or polysubstituted, in particular by C₁-C₄-alkyl, halogen, such as fluorine, chlorine, bromine, halogenated alkyl, such as trifluoromethyl, aryl, such as phenyl, or unsubstituted aryl groups.

The R¹¹ and R¹² radicals may each be separate or bridged. The R²¹ and R²² radicals may also each be separate or bridged. The R¹¹, R¹², R²¹ and R²² radicals may each be separate, two may be bridged and two separate, or all four may be bridged, in the manner described.

In a particularly preferred embodiment, useful compounds are those of the formula I, II, III, IV and V specified in U.S. Pat. No. 5,723,641. In a particularly preferred embodiment, useful compounds are those of the formula I, II, III, IV, V, VI and VII specified in U.S. Pat. No. 5,512,696, in particular the compounds used there in examples 1 to 31. In a particularly preferred embodiment, useful compounds are those of the formula I, II, III, IV, V, VI, VII, VIII, IX, X, XI, XII, XIII, XIV and XV specified in U.S. Pat. No. 5,821,378, in particular the compounds used there in examples 1 to 73.

In a particularly preferred embodiment, useful compounds are those of the formula I, II, III, IV, V and VI specified in U.S. Pat. No. 5,512,695, in particular the compounds used there in examples 1 to 6. In a particularly preferred embodiment, useful compounds are those of the formula I, II, III, IV, V, VI, VII, VIII, IX, X, XI, XII, XIII and XIV specified in U.S. Pat. No. 5,981,772, in particular the compounds used there in examples 1 to 66.

In a particularly preferred embodiment, useful compounds are those specified in U.S. Pat. No. 6,127,567 and the compounds used there in examples 1 to 29. In a particularly preferred embodiment, useful compounds are those of the formula I, II, III, IV, V, VI, VII, VIII, IX and X specified in U.S. Pat. No. 6,020,516, in particular the compounds used there in examples 1 to 33. In a particularly preferred embodiment, useful compounds are those specified in U.S. Pat. No. 5,959,135, and the compounds used there in examples 1 to 13.

In a particularly preferred embodiment, useful compounds are those of the formula I, II and III specified in U.S. Pat. No. 5,847,191. In a particularly preferred embodiment, useful compounds are those specified in U.S. Pat. No. 5,523,453, in particular the compounds illustrated there in formula 1, 2, 3, 4, 5, 6, 7, 8, 9, 10, 11, 12, 13, 14, 15, 16, 17, 18, 19, and 21. In a particularly preferred embodiment, useful compounds are those specified in WO 01/14392, preferably the compounds illustrated there in formula V, VI, VII, VIII, IX, X, XI, XII, XIII, XIV, XV, XVI, XVII, XXI, XXII, XXIII.

In a particularly preferred embodiment, useful compounds are those specified in WO 98/27054. In a particularly preferred embodiment, useful compounds are those specified in WO 99/13983. In a particularly preferred embodiment, useful compounds are those specified in WO 99/64155.

In a particularly preferred embodiment, useful compounds are those specified in the German patent application DE 100 380 37. In a particularly preferred embodiment, useful compounds are those specified in the German patent application DE 100 460 25. In a particularly preferred embodiment, useful compounds are those specified in the German patent application DE 101 502 85.

In a particularly preferred embodiment, useful compounds are those specified in the German patent application DE 101 502 86. In a particularly preferred embodiment, useful compounds are those specified in the German patent application DE 102 071 65. In a further particularly preferred embodiment of the present invention, useful phosphorus chelate ligands are those specified in US 2003/0100442 A1.

In a further particularly preferred embodiment of the present invention, useful phosphorus chelate ligands are those specified in the German patent application of reference number DE 103 50 999.2 of Oct. 30, 2003, which has an earlier priority date but had not been published at the priority date of the present application.

The compounds I, I a, I b and II described and their preparation are known per se. The phosphorus ligands used may also be mixtures comprising at least two of the compounds I, I a, I b and II.

In a particularly preferred embodiment of the process according to the invention, the phosphorus ligand of the nickel(0) complex and/or the free phosphorus ligand is selected from tritolyl phosphite, bidentate phosphorus chelate ligands and the phosphites of the formula I b

P(O—R¹)ₓ(O—R²)_{y}(O—R³)_{z}(O—R⁴)ₚ (I b)

where R¹, R² and R³ are each independently selected from o-isopropylphenyl, m-tolyl and p-tolyl, R⁴ is phenyl; x is 1 or 2, and y, z, p are each independently 0, 1 or 2 with the proviso that x+y+z+p=3; and mixtures thereof.

### Lewis Acid or Promoter

In the context of the present invention, a Lewis acid is either a single Lewis acid or else a mixture of a plurality of, for example two, three or four, Lewis acids.

Useful Lewis acids are inorganic or organic metal compounds in which the cation is selected from the group consisting of scandium, titanium, vanadium, chromium, manganese, iron, cobalt, copper, zinc, boron, aluminum, yttrium, zirconium, niobium, molybdenum, cadmium, rhenium and tin. Examples include ZnBr₂, Znl₂, ZnCl₂, ZnSO₄, CuCl₂, CuCl, Cu(O₃SCF₃)₂, CoCl₂, Col₂, Fel₂, FeCl₃, FeCl₂, FeCl₂(THF)₂, TiCl₄(THF)₂, TiCl₄, TiCl₃, ClTi(O-isopropyl)₃, MnCl₂, ScCl₃, AlCl₃, (C₈H₁₇)AlCl₂, (C₈H₁₇)₂AlCl, (i-C₄H₉)₂AlCl, (C₆H₅)₂AlCl, (C₆H₅)AlCl₂, ReCl₅, ZrCl₄, NbCl₅, VCl₃, CrCl₂, MoCl₅, YCl₃, CdCl₂, LaCl₃, Er(O₃SCF₃)₃, Yb(O₂CCF₃)₃, SmCl₃, B(C₆H₅)₃, TaCl₅, as described, for example, in U.S. Pat. No. 6,127,567, U.S. Pat. No. 6,171,996 and U.S. Pat. No. 6,380,421. Also useful are metal salts such as ZnCl₂, Col₂ and SnCl₂, and organometallic compounds such as RAlCl₂, R₂AlCl, RSnO₃SCF₃ and R₃B, where R is an alkyl or aryl group, as described, for example, in U.S. Pat. No. 3,496,217, U.S. Pat. No. 3,496,218 and U.S. Pat. No. 4,774,353.

According to U.S. Pat. No. 3,773,809, the promoter used may also be a metal in cationic form which is selected from the group consisting of zinc, cadmium, beryllium, aluminum, gallium, indium, thallium, titanium, zirconium, hafnium, erbium, germanium, tin, vanadium, niobium, scandium, chromium, molybdenum, tungsten, manganese, rhenium, palladium, thorium, iron and cobalt, preferably zinc, cadmium, titanium, tin, chromium, iron and cobalt, and the anionic moiety of the compound may be selected from the group consisting of halides such as fluoride, chloride, bromide and iodide, anions of lower fatty acids having from 2 to 7 carbon atoms, HPO₃²⁻, H₃PO²⁻, CF₃COO⁻, C₇H₁₅OSO₂⁻ or SO₄²⁻. Further suitable promoters disclosed by U.S. Pat. No. 3,773,809 are borohydrides, organoborohydrides and boric esters of the formula R₃B and B(OR)₃, where R is selected from the group consisting of hydrogen, aryl radicals having from 6 to 18 carbon atoms, aryl radicals substituted by alkyl groups having from 1 to 7 carbon atoms and aryl radicals substituted by cyano-substituted alkyl groups having from 1 to 7 carbon atoms, advantageously triphenylboron.

Moreover, as described in U.S. Pat. No. 4,874,884, it is possible to use synergistically active combinations of Lewis acids, in order to increase the activity of the catalyst system. Suitable promoters may, for example, be selected from the group consisting of CdCl₂, FeCl₂, ZnCl₂, B(C₆H₅)₃ and (C₆H₅)₃SnX where X═CF₃SO₃, CH₃C₆H₄SO₃ or (C₆H₅)₃BCN, and the preferred ratio specified of promoter to nickel is from about 1:16 to about 50:1.

In the context of the present invention, the term Lewis acid also includes the promoters specified in U.S. Pat. No. 3,496,217, U.S. Pat. No. 3,496,218, U.S. Pat. No. 4,774,353, U.S. Pat. No. 4,874,884, U.S. Pat. No. 6,127,567, U.S. Pat. No. 6,171,996 and U.S. Pat. No. 6,380,421.

Particularly preferred Lewis acids among those mentioned are in particular metal salts, more preferably metal halides, such as fluorides, chlorides, bromides, iodides, in particular chlorides, of which particular preference is in turn given to zinc chloride, iron(II) chloride and iron(III) chloride.

The process according to the invention is associated with a series of advantages. For instance, the hydrocyanation of 3-pentenenitrile with a low degree of conversion is possible without phase separation having to be made possible in the extractive removal of the catalyst system provided by either pre-evaporating 3-pentenenitrile or adding adiponitrile for dilution. The method of hydrocyanation with a low degree of conversion of 3-pentenenitrile which is made possible is associated with a better selectivity of adiponitrile based on 3-pentenenitrile and hydrogen cyanide. The method of hydrocyanation with a low degree of conversion of 3-pentenenitrile which is made possible is additionally associated with a higher stability of the catalyst system.

The optional treatment of the reaction effluent with ammonia or amines and the optional removal of the solids from the reaction effluent allow the process to be optimized further and the separating performance of the extraction to be adjusted.

### EXAMPLES

Percentages specified hereinbelow are percent by mass based on the mixture of adiponitrile (ADN), 3-pentenenitrile (3PN) and the particular ligands. Cyclohexane was not included in the calculation.

### Example I

In a glass flask, 5 g of a mixture (see table for composition) of ADN, 3PN and tritolyl phosphite (TTP) was made up as the ligand under a protective gas atmosphere (argon) and 5 g of cyclohexane were subsequently added. Stirring at a defined temperature achieved mixing of the components. After the stirrer unit had been switched off, the phase separation was monitored visually in the course of continued heating. When two separated phases could not be recognized visually after 5 min, the system was rated as not separated into separate phases. The results are compiled in table 1.

TABLE 1

| | | | Phase | Phase | Phase |
|---|---|---|---|---|---|
| | Ligand | | separation | separation | separation |
| ADN | TTP | 3PN | 20° C. | 40° C. | 60° C. |
| 30% | 0% | 70% | no | no | no |
| 20% | 10% | 70% | no | no | no |
| 40% | 0% | 60% | yes | yes | no |
| 30% | 10% | 60% | yes | no | no |
| 20% | 20% | 60% | no | no | no |
| 40% | 10% | 50% | yes | yes | no |
| 30% | 20% | 50% | yes | no | no |
| 50% | 10% | 40% | yes | yes | yes |
| 30% | 30% | 40% | yes | no | no |
| 50% | 20% | 30% | yes | yes | yes |
| 50% | 30% | 20% | yes | yes | yes |
| 60% | 20% | 20% | yes | yes | yes |

### Example II

The procedure corresponds to that in Example I, except that a chelate ligand of the formula A was used instead of tritolyl phosphite. The results are compiled in table 2.

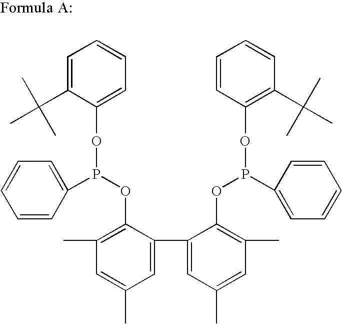

TABLE 2

| | | | Phase | Phase | Phase |
|---|---|---|---|---|---|
| | Ligand | | separation | separation | separation |
| ADN | Formula A | 3PN | 20° C. | 40° C. | 60° C. |
| 30% | 0% | 70% | yes | yes | no |
| 20% | 10% | 70% | no | no | no |
| 40% | 0% | 60% | yes | yes | yes |
| 30% | 10% | 60% | yes | no | no |
| 20% | 20% | 60% | no | no | no |
| 40% | 10% | 50% | yes | yes | yes |
| 30% | 20% | 50% | yes | yes | no |
| 50% | 10% | 40% | yes | yes | yes |
| 30% | 30% | 40% | yes | yes | yes |
| 50% | 20% | 30% | yes | yes | yes |
| 50% | 30% | 20% | yes | yes | yes |
| 60% | 20% | 20% | yes | yes | yes |

### Example III

The procedure corresponded to that in example 1, except that a chelate ligand of the formula B was used instead of tritolyl phosphite. The results are compiled in table 3.

TABLE 3

| | | | Phase | Phase | Phase |
|---|---|---|---|---|---|
| | Ligand | | separation | separation | separation |
| ADN | formula B | 3PN | 20° C. | 40° C. | 60° C. |
| 20% | 10% | 70% | no | no | no |
| 30% | 10% | 60% | yes | yes | no |
| 30% | 20% | 50% | yes | yes | no |
| 60% | 20% | 20% | yes | yes | yes |

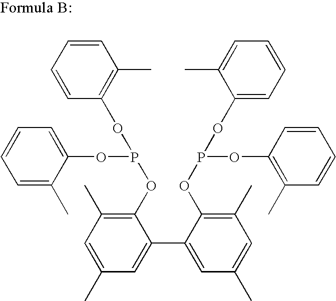

The examples IV and V which follow illustrate the advantageous action of a solids removal.

### Example IV

### Without Solids Removal

4 parts by volume of a mixture of ADN, 3PN and chelate ligand of the formula A were extracted with one part by volume of the hydrocarbon. The hydrocarbon used, the composition of the mixture and the temperature in the extraction and the phase separation can be taken from table 4.

The multiphasic mixtures obtained in the extraction were left to stand in sealed sample vials at a defined temperature. After a certain time, the goodness of the phase separation was determined visually. Table 4 summarizes the results.

TABLE 4
Phase separation

| Composition [% by wt.] | | | | | | | | |
|---|---|---|---|---|---|---|---|---|
| ADN/ | Ligand of | Temp. | Standing | | Phase separation when is used as the hydrocarbon | | | |
| 3PN¹⁾ | formula A | [° C.]²⁾ | time | | Cyclohexane | Methylcyclohexane | n-Heptane | n-Octane |
| 65 | 35 | 23 | 10 | min | no | no | no | no |
| 55 | 45 | 40 | 10 | min | no | no | no | no |
| 40 | 60 | 70 | 2 | min | no | no | Rough | Rough |
| | | | | | | | separation | separation |
| 40 | 60 | 70 | 10 | min | no | no | Rough | Rough |
| | | | | | | | separation | separation |
| 40 | 60 | 70 | 3 | days | Separation, | Separation, | Separation, | Separation, |
| | | | | | but a lot of | but a lot of | a little rag | a little rag |
| | | | | | rag | rag | | |

¹⁾Mixture of 60% by weight of ADN and 40% by weight of 3PN
²⁾Temperature in extraction, phase separation and standing

### Example V

### With Solids Removal

Example V was repeated, but the solids present in the reaction mixture were removed in a decanter before the extraction. The phase separation time until rough separation of the phases was determined. It is compared in table 5 with the separation time of example IV.

TABLE 5
Phase separation times [sec] without solids (example V) and
with solids (example IV) until rough separation; S means solids

| | Hydrocarbon | | | |
|---|---|---|---|---|
| | | Methyl- | | |
| Temperature | Cyclohexane | cyclohexane | n-Heptane | n-Octane |
| 23° C. | | | | |
| without S. | >600 | >600 | >600 | >600 |
| with S. | >600 | >600 | >600 | >600 |
| 40° C. | | | | |
| without S. | >600 | >600 | 150 | 180 |
| with S. | >600 | >600 | >600 | >600 |
| 50° C. | | | | |
| without S. | 180 | 250 | 70 | 70 |
| with S. | >600 | >600 | >600 | >600 |
| 70° C. | | | | |
| without S. | 80 | 80 | 10 | 20 |
| with S. | 300 | 300 | 60 | 100 |

According to this, the phase separation times after removal of the solids were shorter than without solids removal.

The examples VI to IX which follow illustrate the advantageous action of a treatment with ammonia.

### Example VI-a

### Without Ammonia Treatment

In a continuous four-stage mixer-settler extraction apparatus (capacity approx. 150 ml per mixer and settler), a feed was extracted with n-heptane at 40° C. in countercurrent. The feed contained 27.5% by weight of pentenenitrile, 27.5% by weight of adiponitrile and 45% by weight of catalyst, and the catalyst contained the ligands of the formula A, also nickel(0) (in complexed form to the ligand), and finally ZnCl₂, and the molar ratio of these three catalyst components was 1:1:1.

The resulting upper and lower phases were freed continuously of extractant by distillation and this was recycled for the extraction. The apparatus was operated with 100 g/h of feed and 100 g/h of n-heptane until a steady state was attained after 30 hours. Afterward, inputs and outputs were used to conduct a mass balance for one hour under the same conditions.

The mass balance was conducted by using elemental analysis to determine and evaluate the content of phosphorus (as a measure of the phosphorus ligand) and nickel (as a measure of complexed catalyst active component) in the feed and of the collected upper and lower phase obtained. The precision of the mass balance was ±5%, which is why the sum of the percent values of upper and lower phase do not always give precisely 100%.

The mass balances of the examples which follow were conducted in the same manner. Table 6 compiles the mass balances.

### Example VI-b

Example VI-a was repeated, but the molar ratio of the three catalyst components (ligand of the formula A, complexed nickel(0) and ZnCl₂) was 2:1:1.

### Example VII

### With Ammonia Treatment, Without Solids Removal

Example VI-a was repeated, except that the feed was admixed before the extraction in a 4 l round-bottom flask with stirring at 40° C. with 2.2 molar equivalents (based on the ZnCl₂ present) of gaseous, dry ammonia. The ammonia introduced was fully taken up by the solution. After the introduction, any excess ammonia was removed by passing through argon.

In the course of the ammonia introduction, a bright, finely crystalline solid precipitated out which remained in the feed and was also conducted through the extraction. The majority of the solid was discharged from the extraction apparatus with the lower phase; a small portion sedimented and remained in the extraction apparatus.

### Example VIII

### With Ammonia Treatment, with Solids Removal by Filtration

Example VII was repeated, except that the precipitated solid was removed by filtration through a pressure suction filter (depth filter from Seitz, K 700) after the ammonia had been introduced and before the extraction.

### Example IX

### With Ammonia Treatment, with Solids Removal by Decanting

Example VII was repeated; however, the molar ratio of the three catalyst components (ligand of the formula A, complexed nickel(0) and ZnCl₂) was 2:1:1, and the precipitated solids were removed by sedimentation and subsequent decantation after the ammonia had been introduced and before the extraction.

TABLE 6
Mass balance [%] for ligand and nickel(0) (precision ± 5%)

| | Mass balance [%] | | | |
|---|---|---|---|---|
| | Ligand in the | Nickel in the | Ligand in the | Nickel in the |
| Example | upper phase | upper phase | lower phase | lower phase |
| VI-a | 25 | 28 | 72 | 70 |
| VI-b | 51 | 22 | 53 | 76 |
| VII | 99 | 96 | <0.1 | <0.1 |
| VIII | 97 | >99 | <0.1 | <0.1 |
| IX | >99 | >99 | <0.1 | <0.1 |

Examples VI to IX show that the ammonia treatment (examples VIII to IX) distinctly improved the accumulation of ligands and nickel complex in the upper phase. The solids removal before the extraction (examples VIII and IX) allowed the enrichment to be improved once again.

1. A process for extractively removing nickel(0) complexes having phosphorus ligands or free phosphorus ligands from a reaction effluent of a hydrocyanation of unsaturated mononitriles to dinitriles, the process comprising extracting with a hydrocarbon, and separating the hydrocarbon and the reaction effluent into two phases at a temperature T (in ° C.), wherein the content of nickel(0) complexes having phosphorus ligands and free phosphorus ligands in the reaction effluent of the hydrocyanation, depending on the temperature T, is at least y % by weight, where the minimum content y is given by the equation
y=0.5·T+20 and the maximum content of nickel(0) complexes having phosphorus ligands and free phosphorus ligands is 60% by weight.

2. The process according to claim 1, wherein the reaction effluent of the hydrocyanation is treated before or during the extraction with ammonia or a primary, secondary or tertiary aromatic or aliphatic amine.

3. The process according to claim 1, wherein the reaction effluent is treated with anhydrous ammonia.

4. The process according to claim 1, wherein the hydrocarbon used is selected from cyclohexane, methylcyclohexane, n-heptane or n-octane.

5. The process according to claim 1, wherein the hydrocarbon is n-heptane or n-octane.

6. The process according to claim 1, further comprising removing at least a portion of solids present in the reaction effluent before the extraction.

7. The process according to claim 1, wherein the separation of the hydrocarbon is conducted at a temperature of from −15 to 120° C.

8. The process according to claim 1, wherein the extraction provides a high content region in which the content of nickel(0) complexes having phosphorus ligands or free phosphorus ligands is higher than in another region, and the temperature is lower than in the other region.

9. The process according to claim 1, wherein the phosphorus ligand is selected from mono- or bidentate phosphines, phosphites, phosphinites and phosphonites.

10. The process according to claim 1, wherein the phosphorus ligand is selected from tritolyl phosphite, bidentate phosphorus chelate ligands, and phosphites of the formula Ib
P(O—R¹)ₓ(O—R²)_{y}(O—R³)_{z}(O—R⁴)ₚ (I b)
where R¹, R² and R³ are each independently selected from o-isopropylphenyl, m-tolyl and p-tolyl, R⁴ is phenyl, x is 1 or 2, and y, z, p are each independently 0, 1 or 2, with the proviso that x+y+z+p=3; and mixtures thereof.

11. The process according to claim 1, wherein the mononitrile is 3-pentenenitrile and the dinitrile is adiponitrile.

12. The process according to claim 1, wherein the reaction effluent is obtained by reacting 3-pentenenitrile with hydrogen cyanide in the presence of at least one nickel(0) complex having phosphorus ligands,

13. A process for removing nickel(0) complexes having phosphorus ligands and phosphorous compounds from a reaction effluent comprising:
adding a hydrocarbon to the reaction effluent to provide a two phase system in which a first phase is enriched in the nickel(0) complexes having phosphorus ligands and phosphorus compounds and a second phase is enriched with dinitriles at a temperature T (° C.), wherein the maximum concentration of the nickel(0) complexes having phosphorus ligands and phosphorus compounds in the reaction effluent is 60% by weight and the minimum concentration of the nickel(0) complexes having phosphorus ligands and phosphorus compounds is determined by the equation, y=0.5T+20; and
separating the two phase system to provide an isolated first phase and an isolated second phase.

14. The process according to claim 13, wherein the two phase system has an extraction coefficient of 0.8 to 5 as defined by the ratio of mass content of the nickel(0) complexes having phosphorus ligands and phosphorus compounds in the first phase to mass content of the nickel(0) complexes having phosphorus ligands and the phosphorus compounds second phase.

15. The process according to claim 13, wherein the phosphorus ligands and compounds is selected from tritolyl phosphate, bidentate phosphorus chelate ligands, and phosphites of the formula Ib
P(O—R¹)ₓ(O—R²)_{y}(O—R³)_{z}(OR⁴)ₚ
where R¹, R² and R³ are each independently selected from o-isopropylphenyl, m-tolyl and p-tolyl, R⁴ is phenyl, x is 1 or 2, and y, z, p are each independently 0, 1 or 2, with the proviso that x+y+z+p=3; and mixtures thereof.

16. The process according to claim 13, wherein the reaction effluent is produced in a hydrocyanation process for converting 3-pentenenitrile to adiponitrile.

17. The process according to claim 13, wherein the hydrocarbon is selected from cyclohexane, methylcyclohexane, n-heptane or n-octane.

18. The process according to claim 13, further comprising treating the reaction effluent before or during the extraction with ammonia or a primary, secondary or tertiary aromatic or aliphatic amine.
